(19) United States
(12) Patent Application Publication
(10) Pub. No.: US 2025/0231209 A1
(43) Pub. Date: Jul. 17, 2025

(54) METHOD FOR PROVIDING USER INTERFACE AND DEVICE THEREFOR

(71) Applicant: SEEGENE, INC., Seoul (KR)

(72) Inventors: Kwang Il LEE, Seoul (KR); Hye Jin OH, Seoul (KR); Ju Hwa KIM, Gyeonggi-do (KR); Han Deul KIM, Gyeonggi-do (KR); Gyeong Mo GU, Gyeonggi-do (KR); Won Jun KOO, Gyeonggi-do (KR); Eun Ju KANG, Jeollanam-do (KR); Jin Yeong YU, Seoul (KR); Yoon Sik SHIN, Seoul (KR)

(21) Appl. No.: 18/854,613

(22) PCT Filed: Apr. 7, 2023
(86) PCT No.: PCT/KR2023/004750

§ 371 Date: Oct. 7, 2024

(30) Foreign Application Priority Data
Apr. 8, 2022 (KR) 10-2022-0043814

(51) Int. Cl. G01N 35/10; G01N 35/00; G01N 35/04; G06F 3/04847

(52) U.S. Cl. CPC G01N 35/1002; G01N 35/00722; G01N 35/04; G06F 3/04847; G01N 2035/0091; G01N 2035/0493; G01N 2035/1032

(57) ABSTRACT

According to an embodiment of the present invention, there is provided a method method for providing a user interface on a user interface-providing device for operating a liquid handler to prepare a candidate composition to be tested to provide a nucleic acid amplification composition for detecting a target nucleic acid molecule, the method comprising: displaying an input screen for selecting types of tasks and a plurality of test items for preparing the candidate composition: displaying (i) a parameter defined by the selected task and the test item and (ii) an input screen for receiving the parameter value for the displayed parameter; receiving the parameter value; and transferring the received parameter value to the liquid handler, wherein the user interface-providing device communicates with the liquid handler such that the liquid handler is operated at least partially based on an instruction and a parameter value to be referenced upon execution of the instruction, wherein the test of the candidate composition is performed for at least one test item among the plurality of test items, wherein each of the plurality of test items is to test a detection performance of the candidate composition, and wherein the detection performance comprises a sensitivity and/or specificity for the target nucleic acid molecule.

### Technical Field

The present invention relates to a scheme for providing parameter values necessary for operating a preparing device used for preparing a candidate composition to the preparing device.

### BACKGROUND ART

There are various methods of amplifying a target analyte, particularly a target nucleic acid molecule. The methods may include polymerase chain reaction (PCR), ligase chain reaction (LCR) (U.S. Pat. Nos. 4,683,195 and 4,683,202; PCR Protocols: A Guide to Methods and Applications (Innis et al., eds, 1990)), strand displacement amplification (SDA)) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991)), rolling circle amplification (RCA) (Lisby, Mol. Biotechnol. 12(1):75-99 (1999); Hatch et al., Genet. Anal. 15(2):35-40 (1999)) and Q-beta Replicase (Lizardi et al., BiolTechnology 6:1197(1988)), loop-mediated isothermal amplication (LAMP, Y. Mori, H. Kanda and T. Notomi, J. Infect. Chemother., 2013, 19, 404-411), recombinase polymerase amplication (RPA, J. Li, J. Macdonald and F. von Stetten, Analyst, 2018, 144, 31-67).

The above-described amplification reaction requires a detection composition. For example, compositions for nucleic acid detection include, but are not limited to, oligonucleotides (e.g., primers and probes), buffers, enzymes used in amplification reactions, salts, metal ions, dNTPs, and the like.

These detection compositions are materials that have passed various types of tests in laboratories. In other words, a ‘candidate’ composition designed in a laboratory for the detection of a specific target analyte is tested for whether it is suitable for detecting the target analyte through various test processes. Only after passing the test, the ‘candidate composition’ may become a ‘detection composition’ that may be used to detect the target analyte. In the above-described testing process, preparing tasks for a candidate composition are performed. Additionally, these preparing tasks are typically performed in a preparing device designed for these preparing tasks. These preparing units have recently been automated.

### DISCLOSURE OF INVENTION

### Technical Problem

When the above-described preparing device is automated, commands are required to instruct the preparing device what operations to perform. Also required are parameter values that are to instruct the preparing device, e.g., values or directions that are not covered by the commands depending on context.

Therefore, according to an embodiment, there is provided a means or technique for allowing the user of the preparing device to easily input parameter values among the parameter values and commands necessary for the automated preparing device. According to an embodiment, there is provided transferring the input parameter values to the preparing device.

Objects of the disclosure are not limited to the foregoing, and other unmentioned objects would be apparent to one of ordinary skill in the art from the following description.

### Solution to Problem

According to an embodiment, there is provided a method for providing a user interface on a user interface-providing device for operating a liquid handler to prepare a candidate composition to be tested to provide a nucleic acid amplification composition for detecting a target nucleic acid molecule, the method comprising: displaying an input screen for selecting types of tasks and a plurality of test items for preparing the candidate composition; displaying (i) a parameter defined by the selected task and the test item and (ii) an input screen for receiving the parameter value for the displayed parameter; receiving the parameter value; and transferring the received parameter value to the liquid handler, wherein the user interface-providing device communicates with the liquid handler such that the liquid handler is operated at least partially based on an instruction and a parameter value to be referenced upon execution of the instruction, wherein the test of the candidate composition is performed for at least one test item among the plurality of test items, wherein each of the plurality of test items is to test a detection performance of the candidate composition, and wherein the detection performance comprises a sensitivity and/or specificity for the target nucleic acid molecule.

The tasks comprise at least two of a template dilution preparation task, a preparation task of an oligonucleotide mixture, a preparation task of a master mixture comprising an enzyme, a buffer and the oligonucleotide mixture, and an amplification reaction solution setup preparation task for dispensing the master mixture and the template dilution into a container.

If the selected task is a template dilution preparation task, in the input screen for receiving the parameter value is received at least one of a name of the template, a type of a material to be used for diluting the template, a volume of the material to be used for diluting the template, a dilution multiple of the template.

The method further provides a screen displaying at least one of a position where each of a container of the template and a container of the material is placed in a deck of the liquid handler and a position where a container where the template and the material are diluted is placed in a plate of the liquid handler.

If the selected task is a preparation task of an oligonucleotide mixture, in the input screen at least one of names of oligonucleotides, types of the oligonucleotides, concentrations of the oligonucleotides, a dilution factor, and volumes of the oligonucleotides is received.

The method further provides a screen displaying at least one of a position where each of containers of the oligonucleotides is placed in a deck of the liquid handler and a position where a container of the diluted oligonucleotides is placed in a plate of the liquid handler.

If the selected task is an amplification reaction solution setup preparation task, any one step is selected from among the steps of preparing the amplification reaction solution, dispensing the amplification reaction solution, and transferring the template dilution, and wherein if any one step is selected, to the input screen, at least one of a container of the amplification reaction solution, a number of template dilutions, and a number of oligonucleotide mixtures is received.

If the number of template dilutions and the number of the oligonucleotide mixtures are received to the input screen, a position of placement in a plate of the liquid handler is displayed.

Receiving the parameter value includes selecting a parameter value pre-received for at least one test item among the plurality of test items or receiving a new parameter value.

The method of claim 1, wherein the plurality of test items comprises at least one of (i) a sensitivity and specificity when the candidate composition is used to detect the target nucleic acid molecule in different concentrations, (ii) a sensitivity and specificity for a standard microorganism strain to be inclusive of the detection or a microorganism strain to be exclusive of the detection, (iii) a sensitivity and specificity when the candidate composition is contained in each of different container types, (iv) where the candidate composition targets a plurality of target nucleic acid molecules, a sensitivity and specificity when only a single target nucleic acid molecule exists or at least two target nucleic acid molecules exist together and (v) a sensitivity and specificity for each of a clinical sample comprising the target nucleic acid molecule or a clinical sample not comprising the target nucleic acid molecule.

The instruction is defined as a driving action including the actions of moving a pipette module, moving a container, moving a plate, fixing a pipette tip to the pipette module, and inserting the pipette tip into the container or plate to a preset depth, thereby controlling the liquid handler to perform the tasks.

The instruction includes at least one parameter among dispensing or aspirating, a position, a height, a depth, a volume, and a time. The parameter is determined dependent on the instruction.

The liquid handler is an automated liquid handling apparatus capable of automatically and programmatically aspirating and dispensing a desired amount of reagent, sample, or other liquids from a designated container for automation of a chemical or biochemical laboratory.

The type of tasks and the plurality of test item, is displayed simultaneously on the input screen for selecting the type of tasks and the plurality of test item or the task dependent on a selected test item from among the plurality of test items is displayed separately or simultaneously on the screen on which the test item or the type of tasks and plurality of test items are sequentially or randomly displayed.

According to an embodiment, a user interface providing device, comprising: a memory storing at least one instruction; a communication unit performing communication with a candidate composition preparing liquid handler used for testing a candidate composition for providing a nucleic acid amplification composition and operated at least partially based on an instruction and a parameter value to be referenced when the instruction is executed; a display unit; and a processor executing the at least one instruction to control to display an input screen for receiving the parameter value on the display unit, receive the parameter value through the input screen, and control to transfer the received parameter value through the communication unit to the preparing liquid handler, wherein the received parameter value is a value referenced when the preparing liquid handler is operated to prepare the candidate composition.

According to an embodiment, a computer-readable recording medium storing a computer program, the computer program including instructions, when executed by at least one processor, enabling the at least one processor to perform a method performed in a user interface-providing device, targeting the user interface-providing device, wherein the user interface-providing device communicates with a liquid handler used for preparing the candidate composition, and the liquid handler is operated at least partially based on an instruction and a parameter value to be referenced upon execution of the instruction, wherein the method comprising: displaying an input screen for selecting types of tasks and a plurality of test items for preparing the candidate composition displaying (i) a parameter defined by the selected task and the test item and (ii) an input screen for receiving the parameter value for the displayed parameter; receiving the parameter value; and transferring the received parameter value to the liquid handler, wherein the test of the candidate composition is performed for at least one test item among the plurality of test items, wherein each of the plurality of test items is to test a detection performance of the candidate composition, and wherein the detection performance comprises a sensitivity and/or specificity for the target nucleic acid molecule.

### Advantageous Effects of Invention

According to an embodiment, an electronic device is implemented to provide a user interface to allow the user to easily, quickly, and accurately determine values of various types of parameters for operating the preparing device for each preparing task.

### BRIEF DESCRIPTION OF DRAWINGS

FIG. 1 illustrates a device 100 and a liquid handler 200 connected thereto according to an embodiment;

FIG. 2 is a block diagram illustrating a device 100 according to an embodiment;

FIG. 3 illustrates an example screen provided by a device 100 to allow the user of the device 100 to select any one of a plurality of tasks according to an embodiment;

FIG. 4 illustrates an example screen provided by a device 100 to allow the user of the device 100 to select a suitability test for preparing a candidate composition from among a plurality of suitability tests according to an embodiment;

FIG. 5 is a view illustrating an example screen provided to select an option during a detailed task for a reaction solution for amplification according to an embodiment;

FIG. 6 is a view illustrating an example screen for receivng parameter values for a detailed task for amplification reactant setup according to an embodiment;

FIG. 7 is a view illustrating an example parameter value input screen provided when an option for template transfer is selected on the window 510 shown in FIG. 5 during a task for amplification reactant setup according to an embodiment;

FIG. 8 is a view illustrating an example screen for receiving parameter values for an oligonucleotide mixture detailed task according to an embodiment;

FIG. 9 is a view illustrating an example position guide screen for reagents required for preparing an oligonucleotide mixture according to an embodiment;

FIG. 10 is a view illustrating an example screen for receiving parameter values for a template dilution preparation task according to an embodiment; and

FIG. 11 is a flowchart illustrating a method for providing a user interface, on a device according to an embodiment.

### Mode for the Invention

Advantages and features of the present disclosure, and methods for achieving the same may be understood through the embodiments to be described below taken in conjunction with the accompanying drawings. However, the present disclosure is not limited to the embodiments disclosed herein, and various changes may be made thereto. The embodiments disclosed herein are provided only to inform one of ordinary skilled in the art of the category of the present disclosure. The present disclosure is defined only by the appended claims.

When determined to make the subject matter of the present invention unclear, the detailed description of known functions or configurations may be skipped. The terms described below are defined considering the functions in embodiments of the present disclosure and may be replaced with other terms according to the intention or practice of the user or operator. Therefore, the terms should be defined based on the overall disclosure.

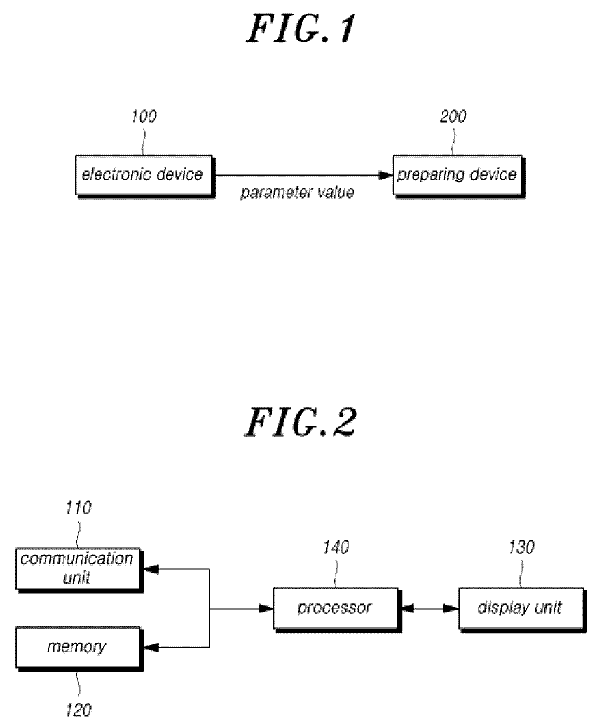

Prior to describing FIG. 1, terms used herein are described.

The term “target analyte” may include a variety of substances (e.g., biological and non-biological substances), which may refer to the same target as the term “target analyte”

Specifically, the target analyte may include a biological substances, more specifically at least one of nucleic acid molecules (e.g., DNA and RNA), proteins, peptides, carbohydrates, lipids, amino acids, biological compounds, hormones, antibodies, antigens, metabolites, and cells.

The term “sample” includes biological samples (e.g., cells, tissues, and body fluids) and non-biological samples (e.g., food, water, and soil). Among them, the biological sample may include at least one of, e.g., virus, germs, tissues, cells, blood (including, e.g., whole blood, plasma, and serum), lymph, bone marrow fluid, saliva, sputum, swab, aspiration, milk, urine, stool, ocular humor, semen, brain extracts, spinal fluid, joint fluid, thymus fluid, bronchoalveolar lavage fluid, ascites, and amniotic fluid. Such sample may or may not include the above-described target analyte.

When the above-described target analyte is or includes a nucleic acid molecule, a nucleic acid extraction process as known in the art may be performed on the sample estimated to contain the target analyte. (See Sambrook, J. et al., Molecular Cloning. A Laboratory Manual, 3^{rd} ed. Cold Spring Harbor Press (2001)). The nucleic acid extraction process may vary depending on the type of sample. Further, when the extracted nucleic acid is RNA, a reverse transcription process may be additionally performed to synthesize cDNA (See Sambrook, J. et al., Molecular Cloning. A Laboratory Manual, 3rd ed. Cold Spring Harbor Press (2001).

The term “data set” refers to the data obtained from a signal generation reaction on the target analyte using a signal generator (the signal generator is described below).

In the case, the term “signal generation reaction” refers to a reaction that generates a signal depending on the properties of the target analyte in the sample, such as activity, amount or presence (or absence), specifically presence (or absence). The signal generation reactions include biological reactions and chemical reactions. Among them, the biological reactions include genetic analysis processes, such as PCR, real-time PCR and microarray analysis, immunological analysis processes, and bacterial growth analysis. Further, the chemical reactions include the process of analyzing the creation, change or destruction of chemicals. According to an embodiment, the signal generation reaction may be a genetic analysis process or may be nucleic acid amplification reaction, enzymatic reaction or microbial growth.

The above-described signal generation reactions are accompanied by signal changes. Therefore, the progress of the signal generation reaction may be evaluated by measuring the change in signal.

Here, the term “signal” means a measurable output. Further, the measured magnitude or change in the signal serves as an indicator qualitatively or quantitatively indicating the properties of the target analyte, specifically, the presence or absence of the target analyte in the sample.

Here, examples of the indicator include fluorescence intensity, luminescence intensity, chemiluminescence intensity, bioluminescence intensity, phosphorescence intensity, charge transfer, voltage, current, power, energy, temperature, viscosity, light scatter, radioactivity intensity, reflectance, transmittance and absorbance, but are not limited thereto.

The term “signal generation means” as described above means a means for providing a signal indicative of the properties of the target analyte to be analyzed, specifically the presence or absence of the target analyte.

The signal generator includes a label itself or a label-attached oligonucleotide.

The label includes a fluorescent label, a luminescent label, a chemiluminescent label, an electrochemical label, and a metal label. The label may be used as a label itself, such as an intercalating dye. Or, the label may be used as a single label or interactive dual label including a donor molecule and an acceptor molecule, in the form attached to one or more oligonucleotides.

When a fluorescent label is used, the signal value may be represented as a relative fluorescence unit (RFU) value.

The signal generator may include an enzyme with nucleolytic activity to generate signals (e.g., an enzyme with 5′ nucleolytic activity or an enzyme with 3′ nucleolytic activity).

Various methods are known for generating a signal indicating the presence of a target analyte, particularly a target nucleic acid molecule, by the signal generator. Representative examples may include: TaqMan™M probe method (U.S. Pat. No. 5,210,015), molecule beacon method (Tyagi, Nature Biotechnology v.14 Mar. 1996), Scorpion method (Whitcombe et al., Nature Biotechnology 17:804-807(1999)), Sunrise or Amplifluor method (Nazarenko et al., Nucleic Acids Research, 25(12):2516-2521(1997), and U.S. Pat. No. 6,117,635), Lux method (U.S. Pat. No. 7,537,886), CPT (Duck P, et al. Biotechniques, 9:142-148(1990)), LNA method (U.S. Pat. No. 6,977,295), Plexor method (Sherrill CB, et al., Journal of the American Chemical Society, 126:4550-4556(2004)), Hybeacons (D. J. French, et al., Molecular and Cellular Probes 13:363-374(2001) and U.S. Pat. No. 7,348,141), Dual-labeled, self-quenched probe; U.S. Pat. No. 5,876,930), hybridization probe (Bernard PS, et al., Clin Chem 2000, 46, 147-148), PTOCE(PTO cleavage and extension) method (WO 2012/096523), PCE-SH(PTO Cleavage and Extension-Dependent Signaling Oligonucleotide Hybridization) method (WO 2013/115442), PCE-NH(PTO Cleavage and Extension-Dependent Non-Hybridization) method (PCT/KR2013/012312) and CER method (WO 2011/037306).

The above-described term “signal generation reaction” may include a signal amplification reaction. In this case, the term “amplification reaction” means a reaction that increases or decreases the signal generated by the signal generator. Specifically, the amplification reaction refers to a reaction for increasing (or amplifying) the signal generated by the signal generation means depending on the presence of the target analyte.

The amplification reaction may or may not be accompanied by amplification of the target analyte (e.g., target nucleic acid molecule). More specifically, the amplification reaction may refer to a signal amplification reaction accompanied by amplification of the target analyte.

The data set obtained through the amplification reaction may include an amplification cycle.

The term “cycle” refers to a unit of change in a predetermined condition in a plurality of measurements accompanied by the change in the condition. The change in the predetermined condition means, e.g., an increase or decrease in temperature, reaction time, number of reactions, concentration, pH, or number of copies of the measurement target (e.g., nucleic acid). Thus, the cycle may be a time or process cycle, unit operation cycle, or reproductive cycle.

More specifically, the term “cycle” means one unit of repetition when a reaction of a predetermined process is repeated or a reaction is repeated based on a predetermined time interval.

Alternatively, the term “cycle” may mean one unit of repetition when a predetermined action is repeated as the reaction proceeds.

For example, when a nucleic acid amplification reaction is performed, the act of detecting a signal generated at regular time intervals may be repeated and may mean one unit of the repetition. In this case, the cycle may have a unit of time.

For example, in the case of a nucleic acid amplification reaction, one cycle means a reaction including denaturation of nucleic acids, annealing of primers, and extension of primers. In this case, the change in the predetermined condition is an increase in the number of repetitions of the reaction, and a repeating unit of the reaction including the above series of steps is set as one cycle. The number of cycles may include the number of reactions or reaction time.

The above-described target analyte, particularly target nucleic acid molecules, may be amplified by various methods: polymerase chain reaction (PCR), ligase chain reaction (LCR) (U.S. Pat. Nos. 4,683,195 and 4,683,202; PCR Protocols: A Guide to Methods and Applications (Innis et al., eds, 1990)), strand displacement amplification (SDA)) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991)), rolling circle amplification (RCA) (Lisby, Mol. Biotechnol. 12(1):75-99 (1999); Hatch et al., Genet. Anal. 15(2):35-40 (1999)) and Q-Beta Replicase) (Lizardi et al., BiolTechnology 6:1197(1988)), loop-mediated isothermal amplication (LAMP, Y. Mori, H. Kanda and T. Notomi, J. Infect. Chemother., 2013, 19, 404-411), recombinase polymerase amplication (RPA, J. Li, J. Macdonald and F. von Stetten, Analyst, 2018, 144, 31-67).

The amplification reaction amplifies the signal while accompanying the amplification of the target analyte (specifically, the target nucleic acid molecule). For example, the amplification reaction is carried out by PCR, specifically real-time PCR, or isothermal amplification reaction (e.g., LAMP or RPA).

The data set obtained by the signal generation reaction include a plurality of data points including cycles of the signal generation reaction and signal values in the cycles.

The term “signal value” refers to a value obtained by quantifying the signal level (e.g., signal intensity) actually measured in a cycle of a signal generation reaction, in particular, an amplification reaction, according to a predetermined scale, or a modified value thereof. The modified value may include a mathematically processed signal value of the actually measured signal value. Examples of mathematically processed signal values of actually measured signal values (i.e., signal values of raw data sets) may include logarithmic values or derivatives.

The term “data point” means a coordinate value including the cycle and signal value. Further, the term “data” refers to all the information constituting the data set. For example, each cycle and signal value of an amplification reaction may correspond to data.

Data points obtained by a signal generation reaction, particularly amplification reaction may be displayed with coordinate values that may be shown in the two-dimensional rectangular coordinate system. In the coordinate values, the X-axis denotes the number of cycles, and the Y-axis denotes the signal value measured or processed in the cycle.

The term “data set” means a set of the data points. For example, the data set may be a set of data points obtained directly through the amplification reaction performed in the presence of the signal generator or a modified data set of such a data set. The data set may be all or some of a plurality of data points obtained by the amplification reaction or modified data points thereof.

The data set may be a data set obtained by processing a plurality of data sets. When analysis on a plurality of target analytes is performed in one reaction vessel, the data set for the plurality of target analytes may, in some cases, be obtained by processing the data sets obtained from the reaction performed in the one reaction vessel. For example, the data set for the plurality of target analytes in the one reaction vessel may be obtained by processing the plurality of data sets obtained from the signals measured at different temperatures.

The above-described data set may be floated, and an amplification curve may thereby be obtained.

The term “plate” refers to a standard unit in which an amplification reaction is performed in an amplification device, and a basic unit in which data generated after an amplification reaction is stored. Different plates may be plates where amplification reactions are performed at different times using the same amplification device or plates where amplification reactions are performed by different amplification devices at the same time.

The plate includes a plurality of reaction wells. The plate may include N×M reaction wells. The plate typically includes 12×8 or 8×12 reaction wells. The reaction well of the plate may be shaped as a tube that is integrated or separable from the plate. The plate may have a rectangular shape. The plate may include one or more reaction wells and may be implemented in various shapes, such as a circle, a trapezoid, or a rhombus, as well as a rectangle.

The well of the plate contains the sample to be analyzed and the reagents necessary for the nucleic acid amplification reaction.

Various embodiments of the present invention are described below with reference to the drawings.

FIG. 1 illustrates a user interface providing device 100 and a preparing device 200 according to an embodiment.

The preparing device 200 is specifically a liquid handler. Liquid handler refers to an automated liquid handling device capable of automatically and programmatically aspirating and/or dispensing desired quantities of reagents, samples or other liquids from designated containers for the automation of chemical or biochemical laboratories. Automated liquid handling devices of various configurations are known to one of ordinary skill in the art. All components of this liquid handler are designed as an integrated device and are positioned within the housing. A pipetting module that may automatically move up and down, left and right, and back and forth is present over the liquid handler deck. The pipetting module may include one or more independently or dependently moving and operating pipettors, i.e., pipette modules. A tip or needle is coupled to the end of the pipettor and used for aspirating and dispensing the solution.

The devices 100 and 200 may be connected to each other by wired or wireless communication. However, the block diagram of FIG. 1 is merely exemplary, and the spirit of the present invention is not limited to those shown in FIG. 1. For example, additional components not shown in FIG. 1 may be connected to the devices 100 and 200. Alternatively, unlike shown, the user interface providing device 100 may be implemented to be included in the liquid handler 200. However, it is assumed that the above-described components 100 and 200 are implemented or connected as shown in FIG. 1. Each component is described below in detail.

The user interface-providing device 100 communicates with the liquid handler 200 used to prepare a candidate composition according to the disclosure.

The liquid handler 200 is operated, at least partially based on the values of the commands and parameter values to be referenced upon execution of the commands.

Based on the same, the liquid handler 200 performs a detection composition preparation process used to detect target nucleic acid molecules in a specimen. The detection composition preparation process may include a process of preparing a reaction solution for nucleic acid extraction and amplification from the specimen.

Further, the liquid handler 200 performs a preparing process of at least one or more candidate compositions for a detection composition necessary for the detection composition preparation process.

In the disclosure, the liquid handler 200 is a device used for preparing candidate compositions.

According to an embodiment, the types of preparing tasks performed in the liquid handler 200 include a template dilution preparation task used for nucleic acid amplification, a task of oligonucleotide mixture, and an amplification reactant task for preparing a master mixture including enzymes and buffers, and the prepared oligo mixture and dispensing the prepared master mixture and the diluted template dilution into containers.

Oligonucleotide mixture preparing task refers to a preparing task of mixing oligonucleotides. Here, oligonucleotides may include forward primers, reverse primers, and probes for each type.

The oligonucleotide mixture preparing task is a task to receive oligonucleotides designed with a desired sequence in one container to detect target nucleic acid molecules. Parameters for the same include the total volume of the oligonucleotide mixture to be prepared, the volume occupied by the oligonucleotide mixture in the master mixture in which the oligonucleotide mixture, TE buffer, and enzyme are mixed, the concentration value for each type of oligonucleotide, the volume of oligonucleotide mixture dispensed into at least one container, a dilution factor that determines a set concentration according to a basic concentration for each oligonucleotide, the name of each oligonucleotide type, the oligonucleotide type for each oligonucleotide name, and the type of the container receiving the same.

The template dilution preparation task used for nucleic acid amplification refers to a task of preparing a template dilution to a desired concentration using a solution, e.g., a buffer. The template may be diluted by adjusting the volume of the buffer from a high concentration to a low concentration. Parameters for the dilution preparation task include concentration multiples, such as 1/10 or 1/100, when diluting the template from a high concentration to a low concentration, template volume for each concentration, and buffer volume for each template concentration. An example of the buffer is a TE buffer.

Next, there is a task of the master mixture including the enzyme, the buffer, and the prepared oligonucleotide mixture. This means preparation of a master mixture for the amplification reaction solution preparation task. During the amplification reaction for the detection of target nucleic acid molecules, various reagents may be added to optimize the reaction, one of which is an enzyme. Upon preparing the master mixture, the enzyme, together with the buffer and the prepared oligonucleotide mixture, is contained in the container. Parameters for the same include the total volume of the master mixture to be prepared, the volume to be aspirated from the container containing each of the oligonucleotide mixture, enzyme, and buffer, and the volume to be dispensed into the container to contain each of the oligonucleotide mixture, enzyme, and buffer as the master mixture, An example of the buffer is a TE buffer.

The amplification reaction solution task includes a task of dispensing the prepared master mixture and the diluted template into a container. If the prepared master mixture and template are received together, a detection composition for the amplification reaction is prepared. Parameters for the same include aspirating and transferring a required volume of template, dispensing it into the container containing the master mixture, and the number of repetitions based on the detection composition to be prepared.

Meanwhile, the candidate composition is a candidate composition proposed in an insilico process. The candidate composition is for selecting the final detection composition. To select the final detection composition from the candidate composition, an optimization process is required to ensure that each of the proposed candidate compositions works well or to make them work well in the insilico process.

According to an embodiment, the liquid handler 200 is a device that performs a preparation task to find out whether the candidate composition works well in the detection process of the target analyte or a preparation task for optimizing the detection of the target analyte. However, the liquid handler 200 is not used only for preparing candidate compositions. For example, the liquid handler 200 may also be used to prepare detection compositions already developed.

However, in the following description, it is assumed that the liquid handler 200 is used to prepare candidate compositions.

According to an embodiment, the liquid handler 200 may be an automated device. In this case, commands, parameters, and parameter values are required for the liquid handler 200 to perform the above-described preparing task. Among them, the commands may be already created and stored in a computer outside the liquid handler 200 or a computer included in the liquid handler or, as necessary, may be newly created. As an example, using a pipettor, which is a pipette module, may include the actions of moving the pipettor module to the fractionation tip, fixing the pipette tip to the pipette module, moving the pipettor with the pipette tip fixed thereto to a certain place, and inserting the pipette tip to a predetermined depth, and the commands allow such actions to be on the liquid handler 200.

In the liquid handler 200 according to the disclosure, the tasks are controlled by software. In other words, the liquid handler 200 may be controlled by computer readable codes executable on a processor or instructions stored on a computer readable recording medium.

Accordingly, the instruction defines minimum unit actions that may occur in the liquid handler 200, and enables the task actions for preparation to be on a combination of the unit actions. As an example, when the liquid handler 200 uses the pipettor, which is a pipette module, there may be minimum basic actions, such as the actions of moving the pipettor to the pipette tip, fixing the pipette tip to the pipettor, moving the pipettor with the pipette tip fixed thereto to a certain place, and inserting the pipette tip to a predetermined depth. Each of the actions is defined as a unit action.

If the task to be executed in the liquid handler 200 is an amplification reaction solution task, task actions, such as the actions of aspirating the master mixture from the container containing the master mixture, dispensing the aspirated master mixture into a specific container, and dispensing the template dilution into the container where the master mixture has been dispensed, should be controlled by the instructions.

The instructions define the actions of moving the pipette module when executed in the liquid handler, moving the receiving container, moving the plate, fixing the pipette tip to the pipette module, and inserting the pipette tip-fixed pipette module to a preset depth and control the liquid handler to perform the tasks.

The instructions include at least one parameter among dispensing or aspirating, position, height, depth, volume, and time, and the parameters are determined dependent on instructions. The instructions selectively combine the unit actions necessary among the above-defined unit actions to allow the task actions for preparation to be executed when executing the task actions. Parameter values are referenced when executing task actions by the instructions.

Parameter value means one referenced when the liquid handler 200 performs a specific operation according to an instruction. The time of performing the specific operation, moving direction, or degree of moving is determined according to the parameter value. For example, the parameter values include the amount dispensed or aspirated when the pipettor performs the action of dispensing or aspirating the liquid, target direction or coordinates of a position when the pipettor performs the action of moving to a specific position, and height when moving up or down the pipettor to a specific height.

Values of these parameters may have various characteristics according to an embodiment. For example, in the task action of moving to a specific reagent position, the parameter value may be a value corresponding to the Dec position ID of the target position. In the template dilution preparation task action, the parameter value may be a volume required for dispensing and aspirating the solution using the pipettor.

According to an embodiment, the user interface-providing device 100 provides the above-described parameter values to the liquid handler 200. To that end, the device 100 receives parameter values from the user of the liquid handler 200. In this case, in an embodiment, the device 100 provides a U/I for receiving parameter values to the user. Accordingly, the user may easily, without error, and accurately input various types of parameter values necessary for operating the liquid handler 200 by using the UI according to the disclosure.

The user interface-providing device 100 is described below.

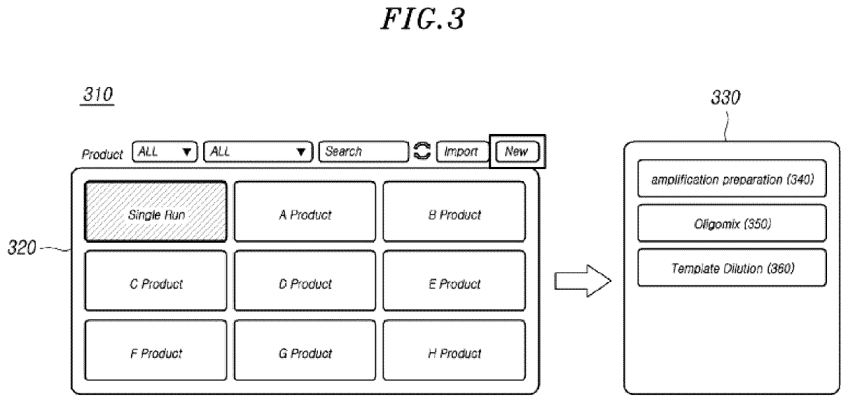

FIG. 2 is a block diagram illustrating a user interface-providing device 100 according to an embodiment.

Referring to FIG. 2, the user interface-providing device 100 may include a communication unit 110, a memory 120, a display unit 130, and a processor 140, but is not limited thereto.

The communication unit 110 is implemented as a wired or wireless communication module. The user interface-providing device 100 may perform communication with the above-described liquid handler 200 through the communication unit 110. For example, as described below, the user interface-providing device 100 may transmit parameter values for each task for the candidate composition received from the user to the liquid handler 200 through the communication unit 120.

The memory 120 stores various types of data including at least one command. As is described below, the stored data may include various types of suitability tests required for the candidate composition task, parameters for each suitability test, and parameter values, but is not limited thereto.

Here, various types of suitability tests include sensitivity and specificity when the candidate composition is received in a 96-well plate and used for detection, sensitivity and specificity when the candidate composition is received in an 8-strip container and used for detection, sensitivity and specificity when the target nucleic acid molecule targeted by the candidate composition and the target nucleic acid molecule not targeted by the data operating circuit DDC are together received in the receiving container of the candidate composition and used for detection, sensitivity and specificity when the candidate composition is used to detect different concentrations of target analytes, sensitivity and specificity when used to detect the strain to be detected by the candidate composition or the strain not to be detected by the candidate composition, and sensitivity and specificity when used to detect the clinical sample to be detected by the candidate composition or the clinical sample not to be detected by the candidate composition.

The memory 120 may store the data processed by the processor 140, and the data may include parameter values necessary for the preparation task, and the parameter values may be stored for each reagent for target nucleic acid molecule.

The display unit 130 displays a screen for receiving parameter values.

In other words, the display unit 130 may be implemented as a means that displays data by the processor 140 and receives data by the processor 140. For example, the display unit 130 may be implemented as a touchscreen or touchpad or may be implemented as a combination of an LCD monitor and a keyboard. The data may be received from the outside through the communication unit 110 by the user interface-providing device 100 or may be loaded from the memory 120, but is not limited thereto.

The processor 140 executes at least one command to control to display an input screen for receiving the parameter values on the display unit 130, receive the parameter values through the display unit 130, and transfer the received parameter values to the preparing device 200 through the communication unit 110.

Here, the received parameter value is a value referenced when the preparing device 200 is operated by instructions for preparation, necessary for testing the candidate composition, as described above.

According to an embodiment, it is on the user interface-providing device necessary for operating the liquid handler that prepares the candidate composition necessary for testing the candidate composition to provide the nucleic acid amplification composition used for detecting the target nucleic acid molecule.

The candidate composition means a test target for whether the detection performance meets a predetermined level or for optimizing the detection performance.

The performance of the final nucleic acid amplification composition selected from among the candidate compositions may determine the result, i.e., whether positive or negative, of determining the presence or absence of the target analyte. the nucleic acid amplification composition is composed of various materials, and the performance of each material needs to be optimized to be selected as the nucleic acid amplification composition for a reagent for determining algorithm detection. Since the nucleic acid amplification composition may exhibit performance differences under various conditions, the task for preparing each material of the nucleic acid amplification composition should be performed very strictly. Further, a performance test as to whether the nucleic acid amplification composition may work well under various conditions should be performed.

The tasks on each material of the nucleic acid amplification composition have conventionally relied on the experimenter's manual work. However, the tasks on each material are performed by the liquid handler 200 which automatically performs the tasks, due to issues, such as human errors in the preparation process and inefficiency in terms of time.

The liquid handler 200 prepares the materials using information, e.g., the type of each material of the nucleic acid amplification composition, concentration, volume, and received position and mixes the materials, preparing the final nucleic acid amplification composition. In this case, the information means parameter values referenced upon executing the instructions for operating the liquid handler 200.

As described above, since the performance of the nucleic acid amplification composition determines the result of determining the presence or absence of the target nucleic acid molecule, it is critical in preparing the nucleic acid amplification composition to prepare at least one candidate composition, set various factors for each candidate composition, and test the same under various conditions to select the optimized nucleic acid amplification composition.

In the disclosure, the parameter value is a factor capable of optimizing the nucleic acid amplification composition and may determine the performance of the candidate composition. This is why it is used to prepare each material of the candidate composition. The materials that make up the candidate composition include oligonucleotide sets (e.g., forward primers, reverse primers, and probes), buffers, enzymes used in amplification reactions, salts, metal ions, dNTPs, and nucleic acid templates. According to an embodiment, the oligonucleotide set may be included for each target nucleic acid molecule to be detected.

The candidate composition according to the disclosure is used to select the detection composition for simultaneously detecting a plurality of target nucleic acid molecules and includes a oligonucleotide set for each target nucleic acid molecule.

The performance of each material reacts sensitively to the parameter values, i.e., the type, volume, and concentration of the container where it is to be received. To optimize the performance of the nucleic acid amplification composition, it is preferable to prepare various candidate compositions while changing the conditions and environments of the materials, and select an appropriate detection composition from the prepared candidate compositions.

Accordingly, in the disclosure, at least one candidate composition for selection as a nucleic acid amplification composition is prepared. Each candidate composition may be a composition in which the materials of the candidate composition are combined in a different volume or concentration.

For example, each candidate composition may be a composition in which in the combination, the volumes or concentrations of the template, buffer, ions, and enzyme are constant for the candidate compositions, but the volume or concentration of the oligonucleotide set is different for each candidate composition. Or, each candidate composition may be a composition in which in the combination, the oligonucleotide set, buffer, ions, and enzyme are the same for the candidate compositions, but only the template differs. As such, different candidate compositions are individually prepared in various combinations of the materials, and a detection composition may be selected from among the so-prepared candidate compositions.

Further, the candidate compositions prepared in different material combinations may exhibit different performances for amplification reaction depending on the types of containers where they are contained. The candidate compositions according to the disclosure are treated as different if contained in different containers although they have the same material combination. The container types include 96 well plate and 8-strip.

Accordingly, parameter values for each material should be set considering various conditions.

For this reason, according to an embodiment, the user interface-providing device 100 may preset, display, and provide parameters necessary for each test to test the prepared candidate compositions under various conditions when preparing at least one candidate composition for the nucleic acid amplification composition using the liquid handler 200. Accordingly, the users may previously receive criteria, i.e., parameters, for the tests necessary for optimizing the performance of the nucleic acid amplification composition to be prepared, upon preparing the candidate compositions.

The user interface-providing device 100 may receive appropriate parameter values from the user for the displayed per-test parameters and transfer the same to the liquid handler 200. Accordingly, the liquid handler 200 performs tasks on the candidate composition based on parameter values according to an embodiment.

According to an embodiment, the user interface-providing device 100 is implemented to provide a user interface for the user of the reagent for target nucleic acid molecule detection to make it possible to quickly and accurately determine the parameter values.

The user may prepare at least one candidate composition to optimize the performance of the nucleic acid amplification composition to be prepared using the user interface-providing device 100.

The user interface-providing device 100 provides a user interface (UI) capable of entry of parameter values for the materials of the candidate composition upon preparing the candidate compositions for selecting the optimized nucleic acid amplification composition. Each functional component of the user interface-providing device 100 providing the UI is described below in detail with reference to FIGS. 3 to 10.

FIG. 3 illustrates an example screen provided by a device 100 to allow the user of the device 100 to select any one of a plurality of tasks according to an embodiment.

The user interface-providing device 100 may display a screen for selecting the task where the parameter values are used. A selection window 330 including the types of tasks may be displayed on the screen.

According to an embodiment, the tasks include at least one of a task for the template dilution used for nucleic acid amplification, a preparation task of an oligonucleotide mixture, a task of a master mixture including an enzyme, buffer, and the prepared oligonucleotide mixture, and an amplification reaction solution setup preparation task for dispensing the prepared master mixture and the diluted template into a container.

In this case, the parameter value is determined depending on the type of selected task.

As shown in FIG. 3, the user interface-providing device 100 displays a screen 310 for selecting a specific task for preparation necessary for a candidate composition test on the display unit 130.

To that end, the liquid handler 200 displays each selection window 330 including the types of tasks for the candidate composition, and each selection window 330 includes the amplification preparation task 330 and the oligonucleotide mixture task 340.

The screen 310 shown in FIG. 3 displays two selection windows 320 and 330. The left-hand selection window 320 of the screen 310 may display the type of the reagent product for target nucleic acid molecule, and the right-hand selection window 330 may display the types of tasks.

Each reagent product for target nucleic acid molecule detection displayed on the left side is to provide, by default, a protocol for, e.g., information used in the process of developing each product, e.g., what enzyme has been used, and what parameter values have been used to prepare each material.

Specifically, if any one of target nucleic acid molecule detection reagent products displayed on the left-hand selection window 320 of the screen 310 is selected by the user, and any one of the types of tasks is selected on the selection window 330 displayed on the right side, the device 100 according to an embodiment may display, for each preparing task, a protocol for, e.g., parameter values used upon preparing each material of the candidate composition and the enzyme used in the process of preparing the nucleic acid amplification composition of the selected target nucleic acid molecule detection reagent product.

The user may refer to the protocol when preparing the candidate composition to be prepared or use the same previous protocol or create a new protocol. In the following description of the device 100 according to the disclosure, an embodiment of receiving per-task parameter values and creating a new protocol is described. However, the disclosure is not limited thereto.

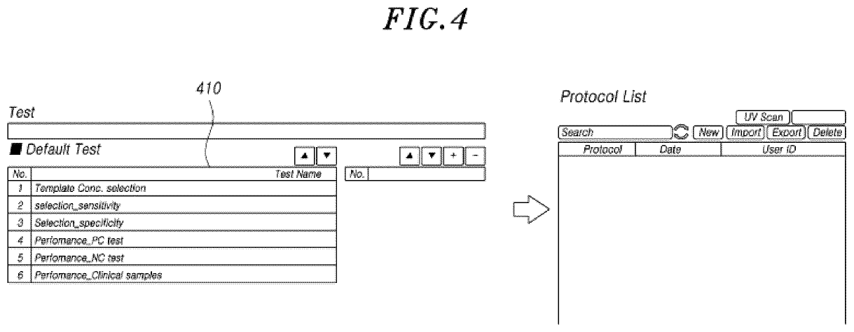

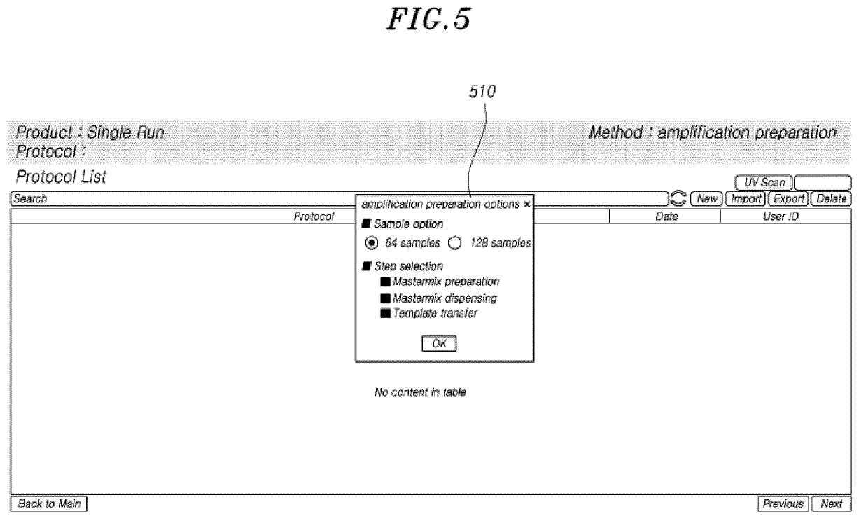

FIG. 4 illustrates an example screen provided by a device 100 to allow the user of the device 100 to select a suitability test for preparing a candidate composition from among a plurality of suitability tests according to an embodiment.

Referring to FIG. 4, the device 100 according to the disclosure receives the user's selection as to what suitability test among a plurality of suitability tests the candidate composition to be prepared in the task selected on the above-described screen 310 is prepared for through the screen 410. Each of the plurality of suitability tests is an experiment to test the performance of the candidate composition. The test of the candidate composition may include a plurality of test items.

The detection performance of the candidate composition may be identified through at least one of the sensitivity and specificity or the sensitivity and specificity when the candidate composition is used for detection.

When a detection channel for each fluorescence wavelength range for qualitative or quantitative analysis of a plurality of target nucleic acid molecules is used, the sensitivity and specificity may be measured for each detection channel.

The detection performance tests of the candidate composition, the plurality of tests displayed on screen 410 as shown in FIG. 4, include at least one of the sensitivity and specificity when the candidate composition is used for detection, the sensitivity and specificity when the candidate composition is received in a different container type, the sensitivity and specificity when only a single standard analyte (e.g., single nucleic acid sequence template) is included or at least two standard analytes (e.g., two or more nucleic acid sequence templates) are together received in a case where the candidate composition in the container containing the candidate composition targets a plurality of standard analytes (e.g., nucleic acid sequence), the sensitivity and specificity when the candidate composition is used to detect different concentrations of target analytes, the sensitivity and specificity for each of the standard microorganism strain to be detected by the candidate composition or the strain to be exclusive of the detection, and the sensitivity and specificity for each of the clinical sample including the standard analyte detected by the candidate composition or the clinical sample not including the detected target analyte.

According to an embodiment, the user may select a specific test item from among the plurality of tests displayed on the screen 410. This is the step of selecting the test for which to use the material to be prepared by the user, e.g., oligonucleotide mixture, and the device 100 according to the disclosure may preset parameters necessary for each test and, if any one test is selected, display and provide the corresponding parameters. The user may input parameter values according to the provided parameters.

According to an embodiment, wherein the type of tasks and the plurality of test item, is displayed simultaneously on the input screen for selecting the type of tasks and the plurality of test item or the task dependent on a selected test item from among the plurality of test items is displayed separately or simultaneously on the screen on which the test item or the type of tasks and plurality of test items are sequentially or randomly displayed.

Preparation of the candidate composition according to the disclosure is part of the development process of a reagent for detecting a target analyte. It is important to select a nucleic acid amplification composition having optimal performance among the candidate compositions. Since the performance of the nucleic acid amplification composition may vary under various conditions, it is necessary to test the performance as to whether each material used in preparing the candidate composition works well under various conditions. However, it may not be easy for non-skilled users to accurately recognize what kind of tests are required in material preparation and what parameters are required for each test.

To that end, in the disclosure, the above-described plurality of tests may be preset when preparing the materials constituting each candidate composition, and parameters necessary for each test may be provided. Thus, the user may prepared the material to be prepared by selecting specific test items from among the plurality of tests.

For example, if the user selects a corresponding test item on the screen 410 according to an embodiment when preparing an oligonucleotide mixture for figuring out the sensitivity and specificity when contained in a 96-well plate and used for detection among a plurality of tests, preset parameters are displayed on the selected test item. Accordingly, the user may input appropriate parameter values to the displayed parameters. In a case where the user performs a template dilution preparation task to figure out the sensitivity and specificity when the candidate composition is received in a 8-strip container and used for detection among the plurality of test items, if the user selects the corresponding test item on the screen 410, preset parameters are displayed on the selected test item, and appropriate parameter values may be input to the displayed parameters. In this case, the parameters are determined depending on the selected experiment.

Meanwhile, the user may refer to the protocol when preparing the candidate composition to be prepared or use it in the same manner or create a new protocol. In the following description of the user interface-providing device 100 according to the disclosure, an embodiment of receiving per-task parameter values and creating a new protocol is described. However, the disclosure is not limited thereto.

Various embodiments for the parameters of each of the plurality of test items and entry of parameter values for each task are described below with reference to the drawings.

First, a screen embodiment for entry of parameter values necessary for the amplification reaction solution setup preparation task in the user interface-providing device 100 is described below.

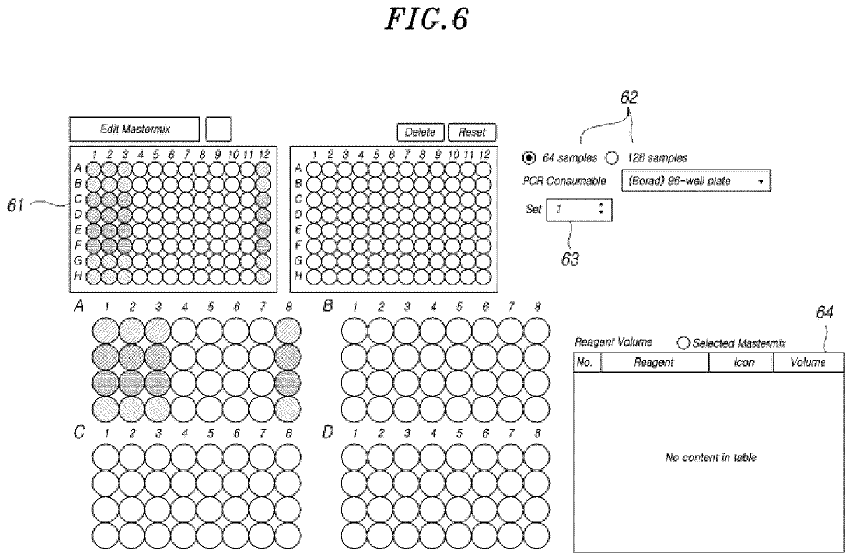

FIG. 5 is a view illustrating an example screen provided to select an option during a task for reaction solution setup for amplification according to an embodiment.

As described above, the amplification reaction solution setup preparation task means preparing a candidate composition by receiving the master mixture and the template together in a container. In the task process, it is required to prepare a master mixture with the oligonucleotide mixture, TE buffer, and enzyme, dispense the prepared master mixture, and transfer the template to the container to which the master mixture is dispensed.

Referring to FIG. 5, when the amplification reaction solution setup preparation task is selected from the selection window 330 of the screen 310 of FIG. 3, the user interface-providing device 100 displays an amplification reaction solution setup preparation task option selection window 510. The selection window 510 displays master mixture prepare, master mixture dispense, and template transfer tasks necessary for the candidate composition task according to an embodiment.

If any one of the tasks is selected, the user may select which suitability test among the plurality of suitability tests the amplification reactant to be prepared is to prepare for on the next screen 410 displayed by the user interface-providing device 100.

If desiring preparation for the purpose of testing the sensitivity and specificity for each of the clinical sample including the target nucleic acid molecule detected by the candidate composition to be prepared or the clinical sample not including the target nucleic acid molecule detected, the user may select a corresponding test item on the screen 410 of FIG. 4.

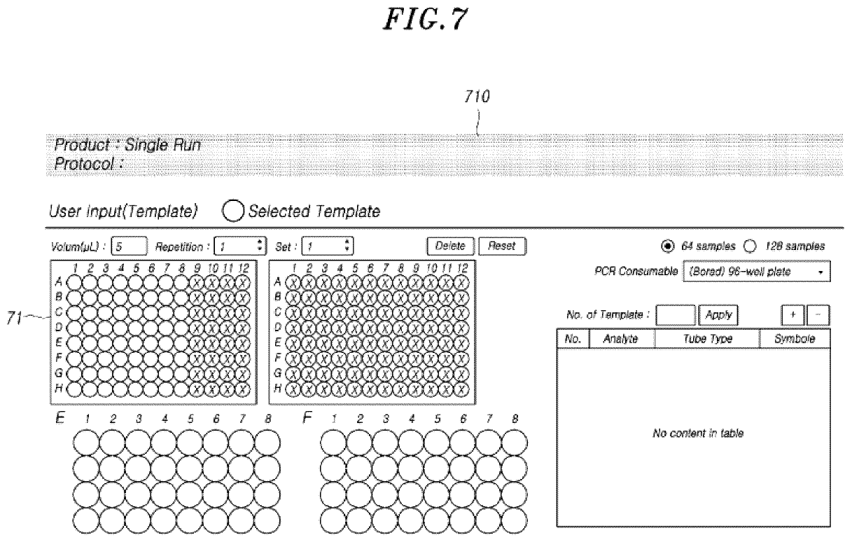

According to an embodiment, the device 100 displays a screen for receiving preset parameters and parameter values to the selected test as shown in FIG. 6.

FIG. 6 is a view illustrating an example screen for receiving parameter values for a preparation task for amplification reaction solution setup according to an embodiment.

When the user desires to prepare the candidate composition for the purpose of testing the sensitivity and specificity when contained in a 96-well plate and used for detection among the plurality of tests, the device 100 according to the disclosure may display a screen as shown in FIG. 6.

Referring to FIG. 6, preparing a master mixture which is an amplification reactant requires the actions of preparing an oligonucleotide mixture, mixing the prepared oligonucleotide mixture and an enzyme and buffer, dispensing the master mixture into a container (e.g., tube), dispensing a template into the master mixture-dispensed container, and transferring the template.

To implement the actions in the liquid handler 200, the device 100 according to the disclosure displays a screen for receiving parameter values as shown in FIG. 6. The parameter values received through the displayed screen are transferred to the liquid handler 200. The liquid handler 200 receiving the parameter values perform the task based on the parameter values.

The parameter values according to the disclosure may have various characteristics. For example, the parameter values may include information about the type, concentration, and volume of the material used for the task of candidate composition, the position where the container containing the material is to be disposed in the deck in the liquid handler, and the position where the container containing the candidate composition is to be disposed in the plate in the liquid handler.

In FIG. 6, the user may enter, e.g., the dispensing position parameter value for receiving the position where the master mixture is to be dispensed in the 96-well plate, the sample count parameter value for receiving the sample count parameter, the container type parameter value for receiving the container type, and the reagent volume parameter value for receiving the dispensed/aspirated amount (volume) for each reagent, such as the oligonucleotide mixture, enzyme, and buffer, upon preparing the master mixture, as parameter values upon preparing the amplification reactant for the purpose of a specific experiment in the amplification reactant task. As such, the parameter values according to the disclosure include the position of a specific well selected from the 96-well plate by the user, the number of samples, the type of the container, the aspirated amount for each reagent, and the dispensed amount of the master mixture.

Meanwhile, according to the disclosure, if the user enters the position where the master mixture is to be dispensed in the 96-well plate 61 on the screen displayed as shown in FIG. 6, the device 100 according to the disclosure may guide to the position where each reagent is to be placed on the deck (A, B, C, D) in the liquid handler 200 for preparing the master mixture based on the position received by the user, and the user may place each reagent to the guided position on the deck in the liquid handler 200.

Further, the user may receive the type of the container to contain the master mixture, as a parameter value, through the container selection field 62 and receive the number of sets (prepared per 96-well plate) of the master mixture to be prepared, as a parameter value, to the set field 63.

Through the screen 610 of FIG. 6, the user may receive the name of each reagent (Oligo, TE buffer, or enzyme) required to prepare the master mixture, and the volume, as parameter values, through the table 64.

The user may designate the position of the well where the master mixture is to be dispensed in the 96-well plate 61 displayed on the screen 610 of FIG. 6 by touch or mouse click. The well designated by the user is received as a parameter value, and is distinguished from the empty well by a color change.

As such, if the 96-well plate 61 position designation parameter for master mixture dispensing is received, the device 100 according to an embodiment may display the position where each reagent (Oligo, TE buffer, and enzyme) is to be placed on the deck (A, B, C, D) in the liquid handler 200 as shown in FIG. 6. Accordingly, the user may position each reagent on deck A in the liquid handler according to the position of each reagent displayed on deck A, as shown in FIG. 6.

Further, the user interface-providing device 100 may guide to the position where the empty container, where each reagent is to be sequentially dispensed, and the master mixture is to be prepared, is to be placed. Based thereupon, the user may sequentially mount each reagent in the position guided to by the user interface-providing device 100 on each deck in the liquid handler 200, and may finally mount the empty container where the reagent is to be mixed in the position guided to by the user interface-providing device 100.

FIG. 7 is a view illustrating an example parameter value input screen provided when an option for template transfer is selected by the user on the amplification preparation task option selection window 510 shown in FIG. 5 during a preparation task for amplification reaction solution setup according to an embodiment.

Referring to FIG. 7, the amplification reaction solution setup preparation task requires a template transfer action. To drive the template transfer action on the preparing device 200, parameter values for the volume of the template, number of repetitions, analyte name, and per-concentration positions of the template are required.

If the user receives the parameter values necessary for the template transfer action through the screen shown in FIG. 7, the liquid handler 200 may refer to the parameter values to perform the template transfer action. For example, the user may receive the position where the template is to be dispensed in the 96-well plate 71, as a parameter value, through the screen 710 of FIG. 7 and, based thereupon, the user interface-providing device 100 according to an embodiment may display the position where the template is to be disposed on the deck (E, F) in the liquid handler 200. The liquid handler 200 transfers the template from the deck according to the volume of template received by the user and performs the action of dispensing the template into a designated well in the 96-well plate 71.

In this case, if the user receives the number-of-repetitions parameter value, as 2, in the repetition field on the screen 710, the preparing device 200 transfers the template to each of two 96-well plates.

In the template transfer action, the template diluted with water may be present for each concentration, and the parameter value for the template transfer action may be received for each template concentration.

Further, the user may receive the type of the container to contain the template, as a parameter value, through the container selection field on the right side of the screen 710 of FIG. 7.

Further, the user may receive per-template names and per-template container types, as parameter values, through the table of the screen 710 of FIG. 7.

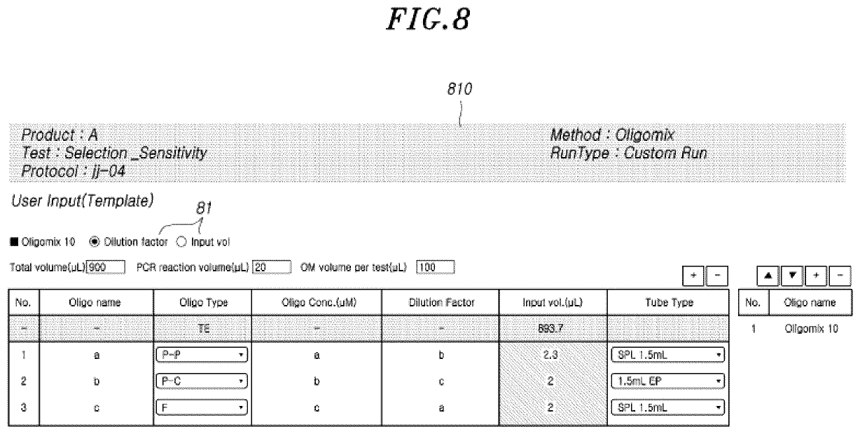

FIG. 8 is a view illustrating an example screen for receiving parameter values for an oligonucleotide mixture task provided by the device 100 according to an embodiment.

Through the screen 810 shown in FIG. 8, the user may select the dilution factor or input volume using the selection field 81 upon preparing the oligonucleotide mixture.

A slight concentration difference exists between the raw materials of oligonucleotides. The concentration difference between the raw materials may be adjusted by a predetermined concentration, i.e., a reference concentration. The dilution factor may be selected to fixedly dispense the input volume based on the reference concentration.

For the input volume, what volume of parameter value each oligonucleotide is to be mixed in may be selected by the user's direct entry.

Based thereupon, the user may receive parameter values necessary upon preparing the oligonucleotide mixture in FIG. 8. The user may receive the total volume ml of the oligonucleotide mixture, the volume of the candidate composition, the proportion ml of the oligonucleotide mixture in the candidate composition, the concentration of the oligonucleotides based thereupon, and the volume of the oligonucleotide mixture, as parameter values, to the respective corresponding parameters.

For example, the user may receive, e.g., the name of the oligonucleotide mixture to be prepared, oligonucleotide type (forward primer, reverse primer, probe, etc.), oligonucleotide mixture total volume, and the type of the container containing the oligonucleotide mixture, as parameter values, through the screen 810 of FIG. 8. The so-received parameter values are parameter values for the test selected as to what purpose the oligonucleotide mixture to be prepared in the plurality of suitability tests is to be used for as described above. Meanwhile, if the above-described parameter values are received, the parameter value for the input volume of the oligonucleotide mixture is automatically calculated.

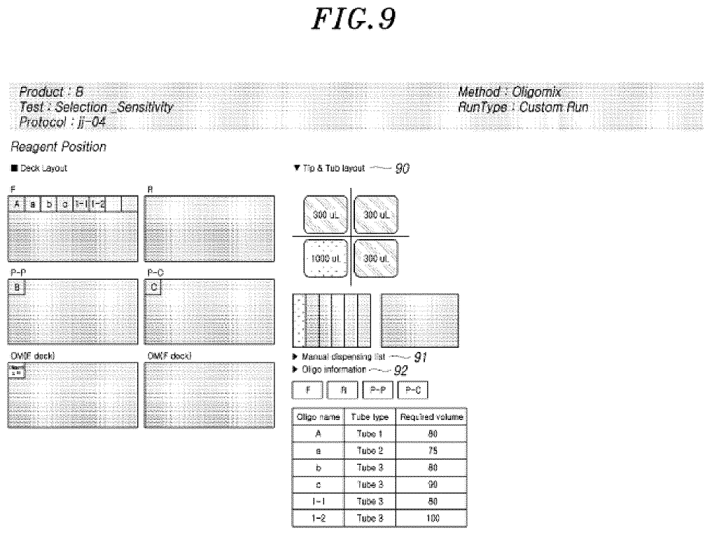

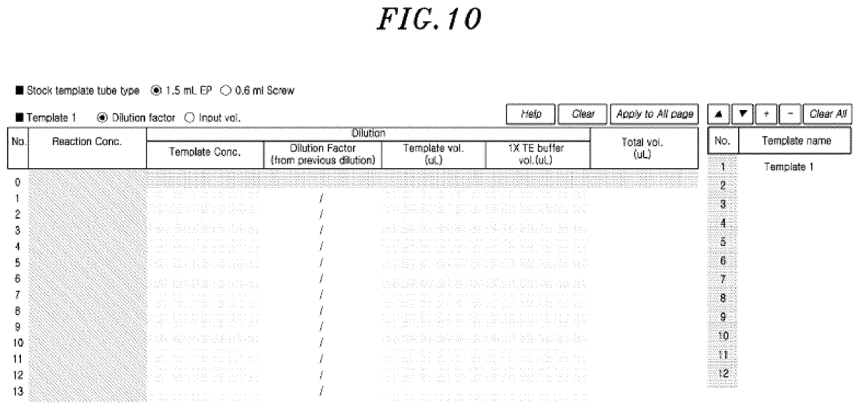

FIG. 9 is a view illustrating an example position guide screen for reagents required for preparing an oligonucleotide mixture in a device 100 according to an embodiment.

If the parameter values for the oligonucleotide mixture task are received by the user through the above-described screen 810, the device 100 according to an embodiment displays the position where each oligonucleotide type (forward primer, reverse primer, and probe (pitcher or catcher)) is to be placed on the deck in the liquid handler 200 to guide the user as shown in FIG. 9.

Further, the device 100 may guide to the position where the empty container, where the forward primer, reverse primer, and probe are to be sequentially dispensed to prepare the oligonucleotide mixture, is to be placed. Based thereupon, the user may sequentially mount the forward primer, reverse primer, and probe in the position guided to by the user interface-providing device 100 on each deck in the liquid handler 200, and may finally mount the empty container where the reagent is to be mixed in the position guided to by the user interface-providing device 100. In this case, the empty containers where the oligonucleotides for each oligonucleotide mixture type are to be finally mixed rely on the parameter value of the number of oligonucleotide mixtures prepared. If the user receives the number-of-oligonucleotide mixtures parameter value as 2, the user interface-providing device 100 guides two mounting positions of two empty containers where the oligonucleotides for each oligonucleotide type are to be finally mixed. Thereafter, as shown in FIG. 9, the user interface-providing device 100 guides to the mounting position for the tip, which is used in the liquid handler 200 to be used to prepare the oligonucleotide mixture, and the mounting position of the tub of the TE buffer, which is used upon preparing the oligonucleotide mixture, through the tip & tub layout field 90. Further, the user interface-providing device 100 may display the parameter values received by the user through the manual dispensing list and oligo information field 91 and 92, together, on the position guide screen for the reagents.

As described above, if the parameter values (e.g., input volume, oligo type, and total volume) for the oligonucleotide mixture to be prepared are received, the device 100 according to an embodiment may visualize the positions of the materials necessary to prepare the oligonucleotide mixture based on the parameter values, thereby eliminating the need for the user to arrange the positions on his own, and further preventing the respective positions of the oligonucleotides from overlapping each other although the number of oligonucleotide mixtures is large.

FIG. 10 is a view illustrating an example screen for receiving parameter values for a template dilution preparation task in a device 100 according to an embodiment.

In FIG. 10, the user may receive parameter values necessary for the template dilution preparation task. Specifically, as shown in FIG. 10, the user may select a dilution factor or an input volume during the template dilution preparation task like in the oligonucleotide mixture preparation.

The dilution factor may be select to set a reference concentration and fixedly dispense the input volume based thereupon. When selecting the dilution factor, a parameter value for the template concentration multiple may be received based on the template concentration. Parameter values for whether to dilute the template from a high concentration to a low concentration by 1/10 or 1/100, the volume of the template, and the TE buffer volume may be received.

For the input volume, what volume of parameter value the template and the TE buffer are to be diluted in may be selected by the user's direct entry. In this case, if the template volume and the TE buffer volume are received, the device 100 according to an embodiment may automatically calculate and display the total volume of them.

Further, the user may receive the container to be used when diluting the template based on such parameter values, as a parameter value, in the displayed stock template tube type field. Further, the user may separate template names according to the number of templates and receive them as parameter values.

Based on such parameter values, the device 100 according to an embodiment visualizes the template container necessary upon diluting the template, and the positions where the container where the template is to be diluted per concentration and contained, and the TE buffer container are to be placed in the preparing device 200.

In the template dilution preparation task, if a high-concentration template is placed, the liquid handler 200 varies the dispensed volume of the TE buffer to adjust the concentration multiple and dispenses the TE buffer along with the template into the empty container, thereby performing the template dilution preparation task. In this case, there may be as many empty containers as the number of templates to be diluted.

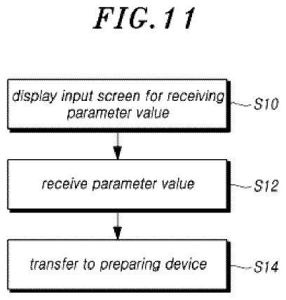

FIG. 11 is a flowchart illustrating a method for providing a user interface, on a device according to an embodiment.

In the disclosure, the user interface-providing device communicates with the liquid handler that prepares a candidate composition necessary for a candidate composition test, and the liquid handler is operated at least partially based on instructions and parameter values to be referenced upon executing the instructions.

Referring to FIG. 11, in S10, the device 100 according to an embodiment displays an input screen for selecting the type of tasks necessary to prepare the candidate composition and a plurality of test items.

In S12, the device 100 according to an embodiment displays the task selected through the screen displayed in S10 and parameters defined by the test items, and displays an input screen for receiving the parameter values for the displayed parameters.

In S14, the device 100 according to an embodiment receives parameter values and transfers the received parameter values to the liquid handler.

The user interface-providing device communicates with the liquid handler for preparing the candidate composition used for the candidate composition test, and the liquid handler is operated at least partially based on instructions and the parameter values to be referenced upon executing the instructions. The candidate composition test is performed by at least one test item among the plurality of test items. Each of the plurality of test items is for testing the detection performance of the candidate composition, and the detection performance of the candidate composition includes the sensitivity and/or specificity for the target nucleic acid molecule.

The per-step operations shown in FIG. 11 are the same as the above-described functions of the user interface-providing device 100, and thus, the description of FIGS. 1 to 10 may be applied thereto.

As described above, the user interface-providing method performed by the device 100 according to an embodiment may provide parameters for each suitability test to optimize the performance of each preparing task for each of the materials used for preparing the nucleic acid amplification composition and provide an interface for receiving the parameter values.

The block diagrams of the disclosure and combinations of the blocks and the steps in the flowcharts may be on a computer program.

A computer-readable recording medium stores a computer program. The computer program includes instructions, when executed by at least one processor, enabling the at least one processor to perform a method performed in a user interface-providing device, targeting the user interface-providing device. The user interface-providing device communicates with a liquid handler used for preparing the candidate composition, and the liquid handler is operated at least partially based on an instruction and a parameter value to be referenced upon execution of the instruction. The method comprises displaying an input screen for selecting types of tasks necessary for preparing the candidate composition and a plurality of test items, displaying a parameter defined by the selected task and the test item and an input screen for receiving the parameter value for the displayed parameter, receiving the parameter value, and transferring the received parameter value to the liquid handler.

Such a computer program may also be stored in a computer-readable recording medium to implement functions by a specific method.

When an element “comprises,” “includes,” or “has” another element, the element may further include, but rather than excluding, the other element, and the terms “comprise,” “include,” and “have” should be appreciated as not excluding the possibility of presence or adding one or more features, numbers, steps, operations, elements, parts, or combinations thereof. All the scientific and technical terms as used herein may be the same in meaning as those commonly appreciated by a skilled artisan in the art unless defined otherwise. It will be further understood that terms, such as those defined dictionaries, should be interpreted as having a meaning that is consistent with their meaning in the context of the relevant art and will not be interpreted in an idealized or overly formal sense unless expressly so defined herein.

The above-described embodiments are merely examples, and it will be appreciated by one of ordinary skill in the art various changes may be made thereto without departing from the scope of the present invention. Accordingly, the embodiments set forth herein are provided for illustrative purposes, but not to limit the scope of the present invention, and should be appreciated that the scope of the present invention is not limited by the embodiments. The scope of the present invention should be construed by the following claims, and all technical spirits within equivalents thereof should be interpreted to belong to the scope of the present invention.

While embodiments of the disclosure have been described above, it will be apparent to one of ordinary skill in the art that the specific techniques are merely preferred embodiments and the scope of the disclosure is not limited thereto. Thus, the scope of the invention is defined by the appended claims and equivalents thereof.

1. A method for providing a user interface on a user interface-providing device for operating a liquid handler to prepare a candidate composition to be tested to provide a nucleic acid amplification composition for detecting a target nucleic acid molecule, the method comprising:
displaying an input screen for selecting types of tasks and a plurality of test items for preparing the candidate composition:
displaying (i) a parameter defined by the selected task and the test item and (ii) an input screen for receiving the parameter value for the displayed parameter;
receiving the parameter value; and
transferring the received parameter value to the liquid handler, wherein the user interface-providing device communicates with the liquid handler such that the liquid handler is operated at least partially based on an instruction and a parameter value to be referenced upon execution of the instruction,
wherein the test of the candidate composition is performed for at least one test item among the plurality of test items, wherein each of the plurality of test items is to test a detection performance of the candidate composition, and wherein the detection performance comprises a sensitivity and/or specificity for the target nucleic acid molecule.

2. The method of claim 1, wherein the tasks comprise at least two of a template dilution preparation task, a preparation task of an oligonucleotide mixture, a preparation task of a master mixture comprising an enzyme, a buffer and the oligonucleotide mixture, and an amplification reaction solution setup preparation task for dispensing the master mixture and the template dilution into a container.

3. The method of claim 1, wherein if the selected task is a template dilution preparation task, in the input screen for receiving the parameter value is received at least one of a name of the template, a type of a material to be used for diluting the template, a volume of the material to be used for diluting the template, a dilution multiple of the template.

4. The method of claim 3, wherein the method further provides a screen displaying at least one of a position where each of a container of the template and a container of the material is placed in a deck of the liquid handler and a position where a container where the template and the material are diluted is placed in a plate of the liquid handler.

5. The method of claim 1, wherein if the selected task is a preparation task of an oligonucleotide mixture, in the input screen at least one of names of oligonucleotides, types of the oligonucleotides, concentrations of the oligonucleotides, a dilution factor, and volumes of the oligonucleotides is received.

6. The method of claim 5, wherein the method further provides a screen displaying at least one of a position where each of containers of the oligonucleotides is placed in a deck of the liquid handler and a position where a container of the diluted oligonucleotides is placed in a plate of the liquid handler.

7. The method of claim 2, wherein if the selected task is an amplification reaction solution setup preparation task, any one step is selected from among the steps of preparing the amplification reaction solution, dispensing the amplification reaction solution, and transferring the template dilution,
and
wherein if any one step is selected, to the input screen, at least one of a container of the amplification reaction solution, a number of template dilutions, and a number of oligonucleotide mixtures is received;

8. The method of claim 7, wherein if the number of template dilutions and the number of the oligonucleotide mixtures are received to the input screen, a position of placement in a plate of the liquid handler is displayed.

9. The method of claim 1, wherein the receiving the parameter value comprisess selecting a parameter value pre-entered for at least one test item among the plurality of test items or receiving a new parameter value.

10. The method of claim 1, wherein the plurality of test items comprises at least one of (i) a sensitivity and specificity when the candidate composition is used to detect the target nucleic acid molecule in different concentrations,
(ii) a sensitivity and specificity for a standard microorganism strain to be inclusive of the detection or a microorganism strain to be exclusive of the detection,
(iii) a sensitivity and specificity when the candidate composition is contained in each of different container types,
(iv) where the candidate composition targets a plurality of target nucleic acid molecules, a sensitivity and specificity when only a single target nucleic acid molecule exists or at least two target nucleic acid molecules exist together and
(v) a sensitivity and specificity for each of a clinical sample comprising the target nucleic acid molecule or a clinical sample not comprising the target nucleic acid molecule.

11. The method of claim 1, wherein the instruction is defined as a driving action including the actions of moving a pipette module, moving a container, moving a plate, fixing a pipette tip to the pipette module, and inserting the pipette tip into the container or plate to a preset depth, thereby controlling the liquid handler to perform the tasks.

12. The method of claim 11, wherein the instruction comprises at least one parameter among dispensing or aspirating, a position, a height, a depth, a volume, and a time, and wherein the parameter is determined dependent on the instruction.

13. The method of claim 1, wherein the liquid handler is an automated liquid handling apparatus capable of automatically and programmatically aspirating and dispensing a desired amount of reagent, sample, or other liquids from a designated container for automation of a chemical or biochemical laboratory.

14. The method of claim 1, wherein the type of tasks and the plurality of test item, is displayed simultaneously on the input screen for selecting the type of tasks and the plurality of test item or
the task dependent on a selected test item from among the plurality of test items is displayed separately or simultaneously on the screen on which the test item or
the type of tasks and plurality of test items are sequentially or randomly displayed.

15. A user interface providing device, comprising:
a memory storing at least one instruction;
a communication unit performing communication with
a candidate composition preparing liquid handler used for testing a candidate composition for providing a nucleic acid amplification composition and operated at least partially based on an instruction and a parameter value to be referenced when the instruction is executed;
a display unit; and
a processor executing the at least one instruction to control to display an input screen for receiving the parameter value on the display unit, receive the parameter value through the input screen, and control to transfer the received parameter value through the communication unit to the preparing liquid handler,
wherein the received parameter value is a value referenced when the preparing liquid handler is operated to prepare the candidate composition.

16. A computer-readable recording medium storing a computer program, the computer program including instructions, when executed by at least one processor, enabling the at least one processor to perform a method performed in a user interface-providing device, targeting the user interface-providing device,
wherein the user interface-providing device communicates with a liquid handler used for preparing the candidate composition, and the liquid handler is operated at least partially based on an instruction and a parameter value to be referenced upon execution of the instruction,
wherein the method comprising:
displaying an input screen for selecting types of tasks and a plurality of test items for preparing the candidate composition
displaying (i) a parameter defined by the selected task and the test item and (ii) an input screen for receiving the parameter value for the displayed parameter;
receiving the parameter value; and
transferring the received parameter value to the liquid handler,
wherein the test of the candidate composition is performed for at least one test item among the plurality of test items,
wherein each of the plurality of test items is to test a detection performance of the candidate composition, and wherein the detection performance comprises a sensitivity and/or specificity for the target nucleic acid molecule.
